(19) United States
(12) Patent Application Publication
(10) Pub. No.: US 2020/0023354 A1
(43) Pub. Date: Jan. 23, 2020

(54) LATERAL FLOW DEVICE

(71) Applicant: Sumitomo Chemical Company Limited, Tokyo (JP)

(72) Inventors: Irene ZACCARI, Godmanchester (GB); May WHEELER, Godmanchester (GB)

(73) Assignee: Sumitomo Chemical Company Limited, Tokyo (JP)

(21) Appl. No.: 16/338,116

(22) PCT Filed: Sep. 28, 2017
(86) PCT No.: PCT/GB2017/052900

§ 371 Date: Mar. 29, 2019

(30) Foreign Application Priority Data
Sep. 29, 2016 (GB) 1616544.1

(51) Int. Cl. B01L 3/00; G01N 33/52; G01N 33/543; G01N 33/558

(52) U.S. Cl. CPC B01L 3/5023; G01N 33/558; G01N 33/54386; G01N 33/528

(57) ABSTRACT

A lateral flow device is disclosed. The device comprises a test layer comprising a test region between opposite first and second end regions of the test layer, a first permeable layer having a first contact area in contact with the first end region, and a second permeable layer having a second contact area in contact with the first end region. The device further comprises a first non-permeable layer between the first and second permeable layers and having a third contact area with the first end region. The first contact area and the second contact area are separated by the third contact area. The device further comprises a sample pad in fluid contact with the first and second permeable layers for delivery of a sample fluid to the first and second contact regions. The second contact area is further from the second end region than the first contact area is from the second end region.

### FIELD OF THE INVENTION

The present invention relates to a lateral flow device.

### BACKGROUND

Biological testing kits may be used for a variety of reasons including, for instance, preliminary diagnosis, screening and management of long term health conditions. Lateral flow devices (also known as “lateral flow immunoassays”) are a variety of biological testing kit. Lateral flow devices may be used to test a liquid sample, such as saliva, blood or urine, for the presence of an analyte. Examples of lateral flow devices include home pregnancy tests, home ovulation tests, tests for other hormones, tests for specific pathogens and tests for specific drugs. For example, EP 0 291194 A1 describes a lateral flow device for performing a pregnancy test.

In a typical lateral flow testing device, a liquid sample is introduced at one end of a porous strip which is then drawn along the strip by capillary action (or “wicking”). A portion of the lateral flow strip is pre-treated with labelling particles which are activated with a reagent which binds to the analyte to form a complex, if the analyte is present in the sample. The bound complexes and also unreacted labelling particles continue to propagate along the strip before reaching a testing region which is pre-treated with an immobilised binding reagent which binds bound complexes of analyte and labelling particles and does not bind unreacted labelling particles. The labelling particles have a distinctive colour, or other detectable optical or non-optical property, and the development of a concentration of labelling particles in the test regions provides an observable indication that the analyte has been detected.

An enzyme-linked immunosorbent assay (“ELISA”) can provide enhancement of a detection signal through the use of enzymes and corresponding substrates. A first portion of the lateral flow strip is pre-treated with enzyme-linked detector particles which are capable of binding with the analyte to form a complex, and a second portion of the lateral flow strip is pre-treated with enzyme substrate particles. A test region is pre-treated with an immobilised binding reagent which binds bound complexes of analyte and enzyme-linked detector particles and does not bind unreacted enzyme-linked detector particles. When the enzyme substrate and the enzyme-linked detector particles mix, a reaction may occur which may result in a change in optical property, for example, a colour change or fluorescence.

If the enzyme substrate and the enzyme-linked detector particles mix in a region outside of the test region, background staining of the lateral flow strip may occur as a result of a reaction between the enzyme and the enzyme substrate. A loss of signal at the test region may also occur.

U.S. Pat. No. 6,706,539 B2 describes an enzyme immunoassay device having a slow lane and a fast lane separated by a hydrophobic barrier, wherein the slow lane contains an enzyme substrate and the fast lane contains an enzyme-antibody conjugate. The fast lane transports fluid at a faster rate than the slow lane. The two ‘lanes’ are fabricated by cutting, aligning, and fusing strips of HDPE. The lanes are aligned along the length parallel to each other.

Mixing of the enzyme and the substrate in the test region can be adversely affected when the pad containing the enzyme and the pad containing the substrate do not contact the test region over the full width of the test region.

### SUMMARY

According to a first aspect of the present invention, there is provided a lateral flow device comprising a test layer comprising a test region between opposite first and second end regions of the test layer. The device comprises a first permeable layer having a first contact area in contact with the first end region and a second permeable layer having a second contact area in contact with the first end region. The device comprises a non-permeable layer between the first and second layers, the non-permeable layer having a third contact area with the first end region. The first contact area and the second contact area are separated by the third contact area. The device comprises a sample pad in fluid contact with the first and second permeable layers for delivery of a sample fluid to the first and second contact regions. The second contact area is further than the first contact area from the second end region.

Thus, the first pad and the second pad can contact the test layer over the full width of the test layer. This can aid mixing of the enzyme and substrate over the full width of the test region. The device can be fabricated by an in-line roll-to-roll process. The device can be fabricated by sheet-by-sheet lamination.

At least a portion of the first non-permeable layer may be interposed between the first permeable layer and the second permeable layer. At least a portion of the second permeable layer may be disposed on the non-permeable layer.

The device may comprise an absorption region in fluid contact with the second end region of the test layer.

The test layer may comprise first and second opposing ends disposed in first and second end regions respectively. The first permeable layer may be in direct contact with the first end.

The first permeable layer may comprise a binding agent linked to a carrier particle. The carrier particle may be a gold nanoparticle. The second permeable layer may comprise a gold-enhancing solution.

An enzyme may be linked to the binding agent. One or more enzymes may be linked to the carrier particle.

The second permeable layer may comprise a gold-enhancing solution or an enzyme substrate. The second layer may comprise a first region proximal to the second contact area and a second region distal to the second contact area. A relatively high concentration of gold-enhancing solution or enzyme substrate may be disposed in the second region of the second layer and a relatively low concentration of gold-enhancing solution or enzyme substrate may be disposed in the first region of the second layer. A thickening agent or a substance capable of slowing fluid flow may be disposed in the first region of the second layer.

The test region may comprise an immobilised specific binding agent. The test region may comprise first and second regions and the immobilised specific binding agent may be disposed in the first region of the test region and an immobilised common binding agent may be disposed in the second region of the test region.

The test layer may comprise a thickening agent or a substance capable of slowing fluid flow disposed between the first contact area and the second contact area.

A path length of the sample fluid within the first permeable layer may be equal to a path length of the sample fluid within the second permeable layer. Thus the first and second permeable layers can be formed by layers having the same dimensions and trimming of the permeable layers may not be required during manufacture.

A path length of the sample fluid within the first permeable layer may be not equal to a path length of the sample fluid within the second permeable layer. Thus, delivery of a substance comprised in the first layer to the test layer or the test region can occur before delivery of a substance comprised in the second layer to the test layer or test region without requiring the second permeable layer to include a substance capable of slowing fluid flow.

The path length may be, for example, a path length between a point of entry into a layer and a point of entry into the test layer. The path length may be a path length between a point of entry into a layer and a point of entry into the test region.

The device may comprise a third permeable layer disposed between the first permeable layer and the first non-permeable layer. The third permeable layer has a fourth contact area in contact with the first end region. The fourth contact area is between the first contact area and the second contact area. The device may comprise a second non-permeable layer disposed between the first permeable layer and the third permeable layer.

A path length of the sample fluid within the third permeable layer may be equal to a path length of the sample fluid within the first permeable layer. A path length of the sample fluid within the third permeable layer may be not equal to a path length of the sample fluid within the first permeable layer.

A path length of the sample fluid within the third permeable layer may be equal to a path length of the sample fluid within the second permeable layer. A path length of the sample fluid within the third permeable layer may be not equal to a path length of the sample fluid within the second permeable layer.

The path length may be, for example, a path length between a point of entry into a layer and a point of entry into the test layer. The path length may be a path length between a point of entry into a layer and a point of entry into the test region.

At least the test layer may be supported by a support sheet.

The sample fluid is preferably a liquid. For example, the sample fluid may be blood, serum, urine, or other biological or non-biological liquid. The permeable layers are preferably permeable to the liquid and the non-permeable layers are preferably non-permeable to the liquid.

According to a second aspect of the present invention there is provided a lateral flow testing kit comprising a device according to the first aspect and a housing, wherein the device is received in the housing.

The housing may comprise a base portion, and a lid portion comprising a first aperture and a second aperture, and the device may be received such that at least a part of the sample pad is accessible through the first aperture and at least a part of the test region is accessible through the second aperture.

According to a third aspect of the present invention there is provided a method comprising providing a device according to the first aspect or a kit according to the second aspect, providing a fluid sample, and applying the fluid sample to the sample pad.

According to a fourth aspect of the present invention there is provided a method of delivering a gold-enhancing solution to a test region comprised in a lateral flow device after gold nanoparticles are delivered to the test region.

According to a fifth aspect of the present invention there is provided a lateral flow device configured to deliver a gold-enhancing solution to a test region comprised in the lateral flow device after gold nanoparticles are delivered to the test region.

### BRIEF DESCRIPTION OF THE DRAWINGS

Certain embodiments of the present invention will now be described, by way of example, with reference to the accompanying drawings, in which:

FIG. 1 is a perspective view of a first lateral flow device;

FIG. 2 is a perspective view of a first lateral flow device showing areas of contact within the device;

FIG. 3 is a cross-sectional view of a first lateral flow device;

FIG. 4 is a plan view of a first face of a test layer comprised in a first lateral flow device showing contact areas on the first face;

FIGS. 5a to 5f illustrate propagation of a sample fluid through a first lateral flow device;

FIG. 6 is a cross-sectional view of a first end of a first lateral flow device;

FIG. 7 is a cross-sectional view of a second permeable layer comprised in a first lateral flow device;

FIG. 8 is a cross-sectional view of a first end of a first lateral flow device;

FIG. 9 is a cross-sectional view of a first modified lateral flow device;

FIG. 10 is a plan view of a first face of a test layer comprised in a first modified lateral flow device showing contact areas on the first face;

FIG. 11 is a cross-sectional view of a second modified lateral flow device;

FIG. 12 is a plan view of a first face of a test layer comprised in a second modified lateral flow device showing contact areas on the first face;

FIG. 13 is a cross-sectional view of a second lateral flow device; and

FIG. 14 is a cross-sectional view of a lateral flow device held in a housing.

### Detailed Description of Certain Embodiments

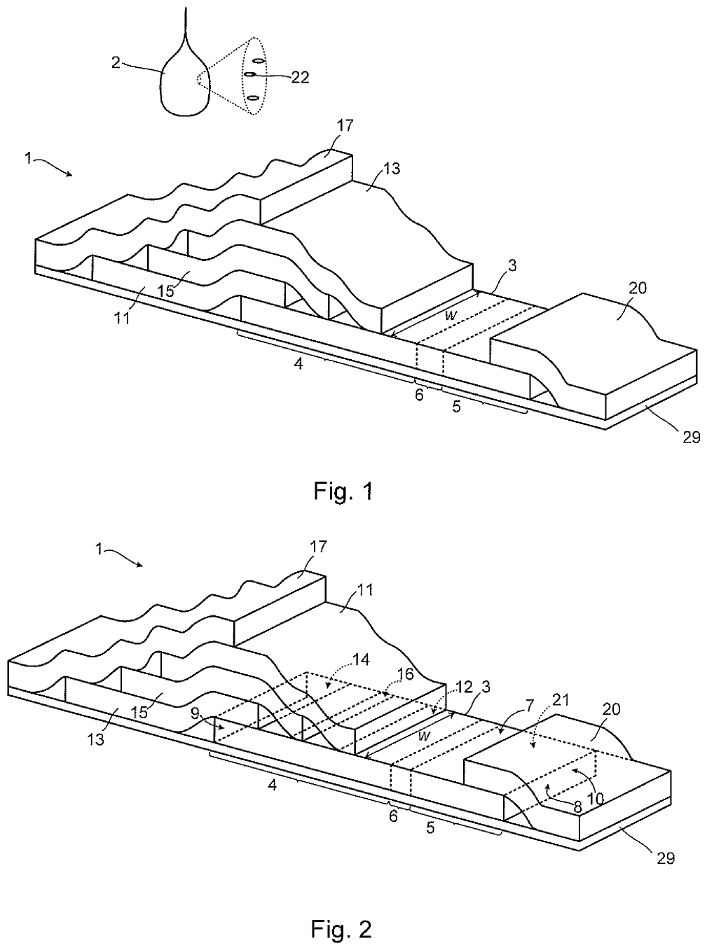

Referring to FIGS. 1 to 3, a device 1 is shown for testing a liquid sample 2.

The liquid sample 2 is a sample suspected of including an analyte. The analyte may be an antigen, a hormone, an enzyme, a protein, an immunoglobulin, DNA, RNA, glucose, lactose, or any analyte which is capable of being detected using the methods described herein. The liquid sample 2 may be blood, urine, serum, saliva, or other biological or non-biological fluid.

The device 1 comprises a generally elongate, strip-like test layer 3. The test layer 3 includes first and second end regions 4, 5 and a test region 6 between first and second end regions 4, 5.

The test layer 3 has first and second opposite faces 7, 8 and first and second opposite end faces 9, 10, herein referred to as first and second ends 9, 10. The first and second opposite ends 9, 10 are part of first and second end regions 4, 5. The test layer 3 has a length l measured between the first and second ends 9, 10 and a width w measured perpendicular to the length l in the plane of the first face 7.

The test layer 3 can hold and transport the liquid sample 2. The test layer 3 may transport the liquid sample 2 by capillary action (also known as “wicking”). The test layer 3 may be made from one or a combination of materials such as, for example, cellulose filter, nitrocellulose, polyvinylidene fluoride, polyethersulfone (PES), charge modified nylon or surface modified polyester.

The device 1 includes a first permeable layer 11 having a first contact area 12 in contact with the first face 7 in the first end region 4. The first permeable layer 11 is permeable to the liquid sample 2. The first contact area 12 is in contact with the first face 7 across the full width w of the test layer 3, that is, the width of the first contact area 12 is equal to w.

The first permeable layer 11 comprises one or more permeable materials such as glass fibre, rayon, polyester or cellulose.

The first permeable layer 11 contains an enzyme-linked binding agent 23 comprising a binding agent 24 which is linked, conjugated, or otherwise coupled to an enzyme 25. The binding agent 24 is capable of binding the analyte 22 specifically. For example, if the analyte 22 is an antigen, the binding agent 23 may be chosen to be an antibody, or aptamer, which binds the antigen. The enzyme 25 may be, for example, horseradish peroxidase, alkaline phosphatase, urease.

The first permeable layer 11 may include one or more materials which can perform one or more functions such as optimising a chemical reaction between the analyte 22 and the binding agent 24, stabilising the enzyme-linked binding agent 23, ensuring efficiency of release of the enzyme-linked binding agent 23 from the first permeable layer 11. For example, the first layer may include a salt-sugar matrix or a polymer. The first permeable layer may include one or more of a protein, a surfactant, a solubilisation agent. A surfactant can aid release and fast flow of the enzyme-linked binding agent 23 from the first layer 11 when a sample fluid 2 flows through the first layer 11.

The device 1 comprises a second permeable layer 13 having a second contact area 14 in contact with the first end region 4. The second permeable layer 13 is permeable to the liquid sample 2. The second permeable layer 13 is in contact with the first end 9 of the test layer 3. The second contact area 14 is in contact with the first face 7 across the full width w of the test layer 3, that is, the width of the second contact area 14 is equal to w.

The second permeable layer 13 comprises one or more permeable materials such as glass fibre, rayon, polyester.

The second permeable layer 13 contains an enzyme substrate 26. The enzyme substrate 26 is a substrate capable of reacting with the enzyme 25 linked to the binding agent 24 contained in the first permeable layer 11. For example, one substrate capable of reacting with horseradish peroxidase is 3,3′,5,5′-Tetramethylbenzidine (TMB). The enzyme substrate 26 is preferably a chromogenic substrate.

The second permeable layer 13 may include one or more materials which can perform one or more functions such as ensuring efficiency of release of the enzyme substrate 26 from the second permeable layer 13, decreasing a propagation speed of a fluid through the second permeable layer 13 relative to a propagation speed of a fluid through the first permeable layer 11. For example, the second permeable layer 11 may include one or more of a sugar, a salt, a pH buffer, a protein, a thickening agent such as sucrose or maltose, a solubilisation agent such as polysorbate or polyethylene glycol, or other detergent or surfactant.

The device 1 comprises a first non-permeable layer 15 between the first permeable layer 11 and the second permeable layer 13. The first non-permeable layer 15 is non-permeable to the liquid sample 2. The first non-permeable layer 15 has a third contact area 16 with the first end region 4. The first contact area 12 and the second contact area 14 are separated by the third contact area 16. The first non-permeable layer 15 may comprise a plastic material such as HDPE, PTFE, PP, PVP, PVC, polyester, polystyrene.

The device 1 comprises a sample pad 17 in fluid contact with the first permeable layer 11 and the second permeable layer 13. The sample pad 17 has a first interface 18 with the first permeable layer 11 and a second interface 19 with the second permeable layer 13. The sample pad 17 comprises one or more absorbent materials such as cotton linter, high density cellulose, glass fibre, rayon, polyester. The sample pad 17 may include one or more of a pH buffer, a surfactant, a blocking reagent.

The device 1 comprises an absorbent pad 20 having a fourth contact area 21 in contact with the second end region 5. The absorbent pad 20 is in contact with the second end 10 of the test layer 3. The fourth contact area 21 is in contact with the first face 7 across the full width w of the test layer 3, that is, the width of the fourth contact area 21 is equal to w.

The absorbent pad 20 comprises one or more absorbent materials such as cotton linter or cellulose. The absorbent pad 20 functions to draw sample fluid through the test layer 3 and functions as a container for fluid drawn through the test layer 3. The absorbent pad 20 is preferably capable of absorbing the entire volume of an applied sample fluid 2 so as to allow the entire volume of the fluid 2 to be drawn through the test layer 3. The absorption capacity of the absorbent pad 20 may be controlled by choice of materials used in the absorbent pad and/or choice of dimensions of the pad.

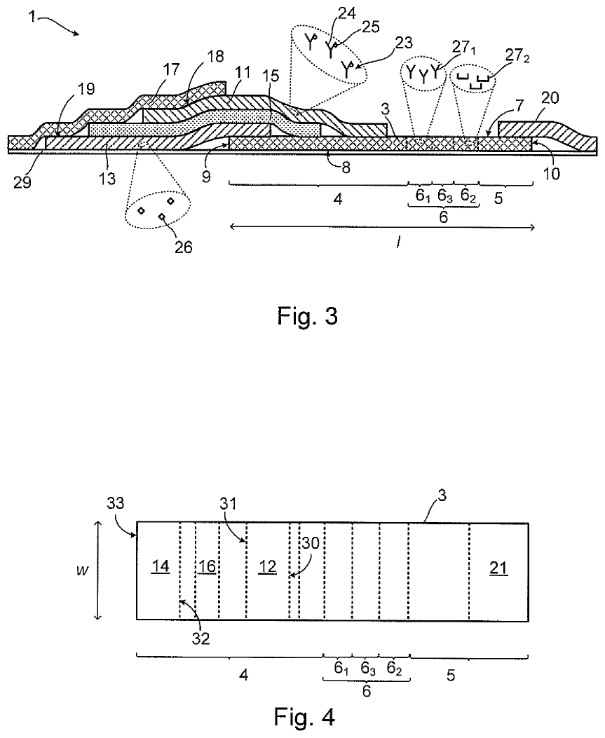

Referring in particular to FIG. 3, the test region 6 includes a first region 6, containing an immobilised specific binding agent 27₁. The immobilised binding agent 27₁ is capable of binding the analyte 22 specifically. For example, if the analyte 22 is an antigen, the immobilised binding agent 27₁ may be chosen to be an antibody which binds the antigen.

The test region 6 includes a second region 6₂, which may also be referred to as a control region, containing an immobilised common binding agent 27₂. The immobilised common binding agent 27₂ is capable of binding the enzyme-linked binding agent 23. For example, the immobilised common binding agent 27₂ may be an antibody or aptamer specific to the binding agent 23, or a nucleic acid sequence complementary to a nucleic acid binding agent.

The first region 6₁ and the control region 6₂ are separated by a third region 6₃.

The device 1 is supported by a support sheet 29. The support sheet 29 may comprise one or more of PVC, card, polyester, polystyrene. The device 1 may be attached to the support sheet 29 by an adhesive (not shown). The device 1 may be laminated to the support sheet 29.

Referring to FIG. 4, the first contact area 12, the second contact area 14, and the third contact area 16 are disposed in the first end region 4 of the test layer 3. The third contact area 16 is between the first contact area 12 and the second contact area 14. The first contact area 12 is further than the second contact area 14 from the second end region 5. The fourth contact area 21 is in the second end region 5 of the test layer 3.

The first contact area 12 has a proximal end 3o and a distal end 31. The second contact area 14 has a proximal end 32 and a distal end 33. The proximal end 32 of the second contact area 14 is further from the second end region 5 than the distal end 31 of the first contact area 12 is from the second end region 5.

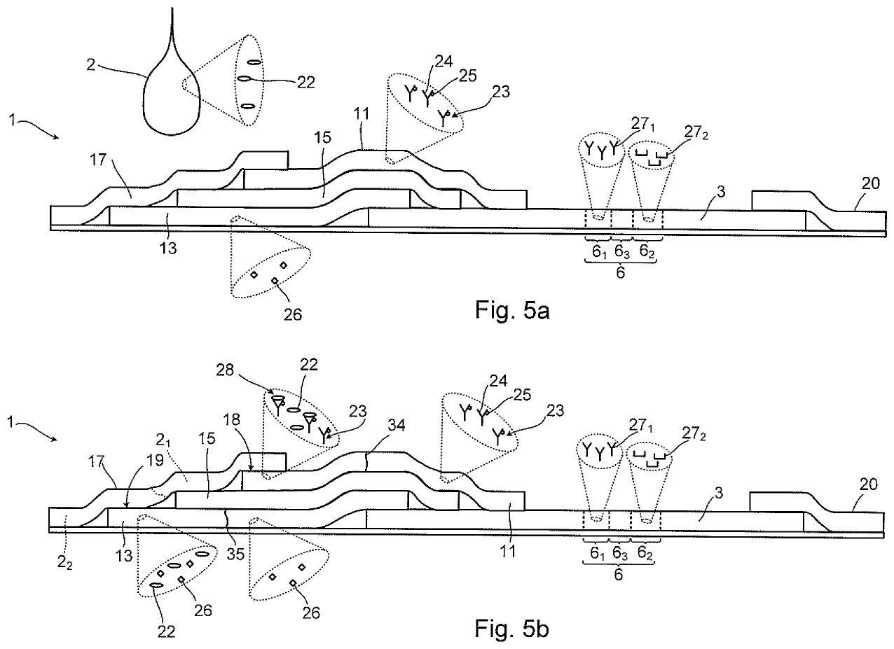

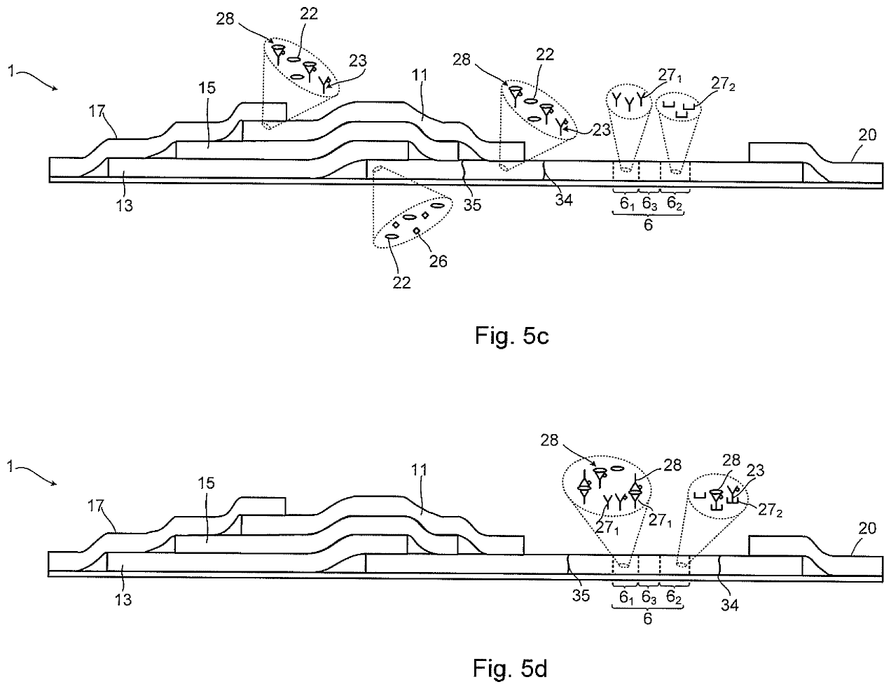

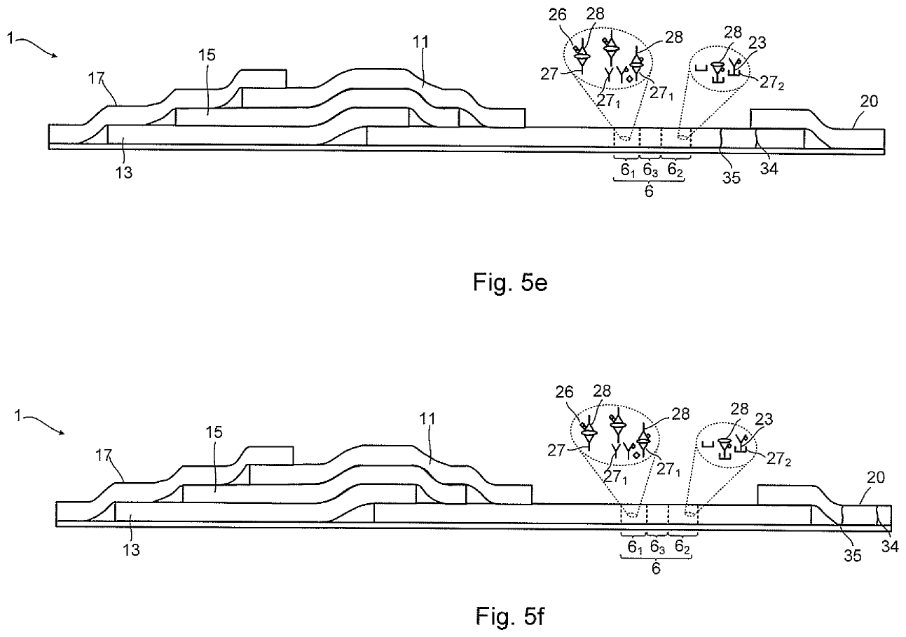

Fluid propagation through the lateral flow device 1 will now be described with reference to FIGS. 5a to 5f.

Referring to FIG. 5a, sample fluid 2, which is suspected of containing an analyte 22, is applied to the sample pad 17. The sample pad 17 absorbs the sample fluid 2. The sample fluid 2 may dissolve or disperse one or more materials contained by the sample pad 17, for example, a pH buffer or a surfactant. The materials may react with components of the sample fluid and additionally or alternatively may be transported by the fluid 2.

Referring to FIG. 5b, sample fluid 2 propagates through the sample pad 17 and contacts the first and second permeable layers 11,13 at first and second interfaces 18, 19 respectively.

A first portion 2₁ of sample fluid 2 is absorbed by and propagates through the first permeable layer 11. The first fluid portion 2₁ has a first flow front 34. The first fluid portion 2₁ dissolves or disperses enzyme-linked binding agents contained in the first layer 11. If the first fluid portion 2₁ contains an analyte 22 which is capable of binding to the enzyme-linked binding agent 23, then a binding reaction may occur between the analyte 22 and the enzyme-linked binding agent 23. The first fluid portion 2₁ may then transport enzyme-linked bound complexes 28 comprising an enzyme-linked binding agent 23 bound to an analyte 22.

A second portion 2₂ of sample fluid 2 is absorbed by and propagates through the second permeable layer 13. The second fluid portion 2₂ dissolves or disperses enzyme substrates 26 contained in the second permeable layer 13. The second fluid portion 22 has a second flow front 35.

Referring to FIG. 5c, the first fluid portion 2₁ propagates through the first permeable layer 11 to the first contact area 12 and is drawn into the test layer 3. The first fluid portion 2₁ propagates through the test layer 3 towards the absorbent pad 20. The first fluid portion 2₁ may transport unbound analyte 22, enzyme-linked bound complexes 28, and enzyme-linked binding partners 23.

The second fluid portion 2₂ propagates through the second permeable layer 13 to the second contact area 14 and is drawn into the test layer 3. The second fluid portion 2₂ propagates through the test layer 3 towards the absorbent pad 20. The second fluid portion 2₂ may transport analyte 22 and substrate 26.

Referring to FIG. 5d, when the first fluid portion 2₁ propagates through the first region 6₁ of the test region 6, an enzyme-linked bound complex 28 transported by the first fluid portion 2₁ may bind to an immobilised specific binding agent 27₁ in the first region 6₁ of the test region 6 and cease to be transported by the first fluid portion 2₁. When the first fluid portion 2₁ propagates through the control region 6₂ of the test region 6, an enzyme-linked binding partner 23 may bind to an immobilised common binding agent 27₂ in the control region 6₂ of the test region 6 and cease to be transported by the first fluid portion 2₁.

Referring to FIG. 5e, when the second fluid portion 2₂ propagates through the first region 6₁ of the test region 6, an enzyme substrate 26 transported by the second fluid portion 2₂ may react with an enzyme-linked bound complex 28 bound to an immobilised specific binding agent 27₁ in the first region 6₁ of the test region 6 and cease to be transported by the second fluid portion 2₂. The reaction between an enzyme substrate 26 and an enzyme-linked bound complex 28 may cause, increase, or enhance a colour change in the test layer. The reaction may cause emission of fluorescence (not shown).

Binding of an enzyme-linked binding partner 23 to an immobilised common binding agent 27₂ in the control region 6₂ may occur in the absence of analyte 22. Binding of an enzyme-linked bound complex 28 to an immobilised specific binding agent 27₁ in the first region 6₁ does not occur in the absence of analyte 22. Thus, the control region 6₂ may be considered to be providing an indication of propagation of the fluid 2 through the test layer 3 regardless of the presence of the analyte 22.

Referring to FIG. 5f, the first and second fluid portions 2₁, 2₂ are drawn into the absorbent pad 20.

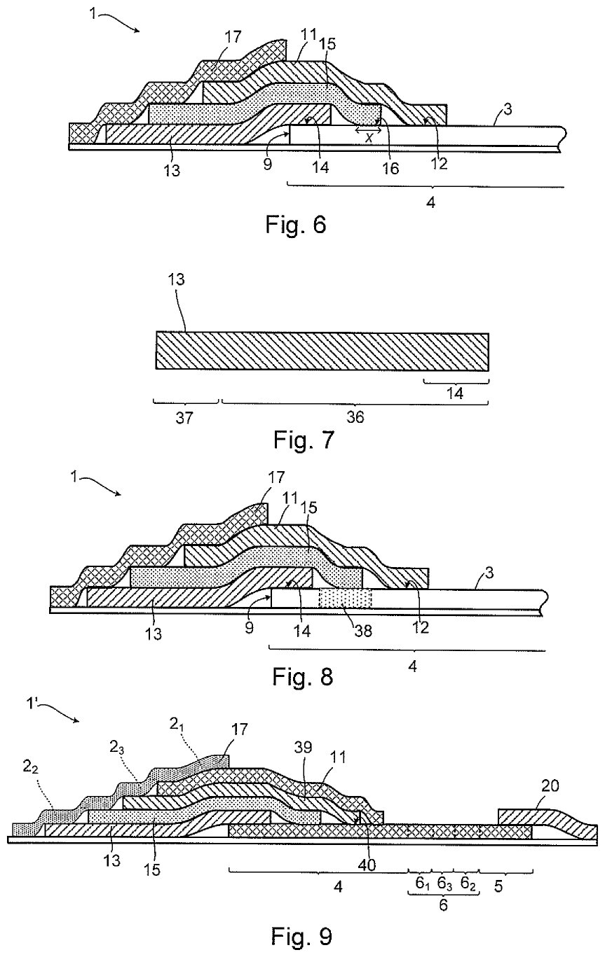

Referring to FIG. 6, the third contact area 16 has a length x in the direction of flow of the sample fluid 2 and the first contact area 12 is closer than the second contact area 14 to the absorbent pad 20. Thus, the first fluid portion 21 enters the test layer 3 at a distance from the test region 6 which is at least x closer than a point at which the second fluid portion 2₂ enters the test layer 3.

This can result in propagation of the first flow front 34 through the test region 6 before the second flow front 35 propagates through the test region 6. Thus, when the second flow front 35 reaches the test region 6, at least a portion of the enzyme-linked bound complex 28 transported by the first fluid portion 2₁ may be bound to the immobilised specific binding agent 27₁ in the first region 6₁ of the test region 6. A colour change may be observed in the test region 6 as a result of a reaction between a substrate 26 and an enzyme-linked bound complex 28 bound to an immobilised specific binding agent 27₁.

The first permeable layer 11 may comprise a material having a wicking rate which is faster than the wicking rate of the material of the second layer 13. For example, the first permeable layer 11 may comprise a first material having a first pore size and the second permeable layer 13 may comprise a second material having a second pore size and the first pore size may be smaller than the second pore size.

This can help to increase the flow rate of the first fluid portion 21 through the first permeable layer 11 relative to the flow rate of the second fluid portion 22 through the second permeable layer 13. This can help to increase time between entry of the first fluid portion 21 into the test layer 3 and entry of the second fluid portion 22 into the test layer 3.

The second permeable layer 13 may include a thickening agent. This can slow the flow rate of the second fluid portion 22 through the second permeable layer 13 and further increase time between entry of the first fluid portion 21 into the test layer 3 and entry of the second fluid portion 22 into the test layer 3.

Referring to FIG. 7, the second permeable layer 13 may comprise a first region 36 proximal to the second contact area 14 and a second region 37 distal to the second contact area 14. The enzyme substrate 26 may be distributed such that there is a is relatively high concentration of enzyme substrate 26 in the second region 37 and a relatively low concentration of enzyme substrate 26 in the first region 36, for example, no enzyme substrate 26 is disposed in the first region 36. A thickening agent (not shown) may be disposed in the first region 36. When the second fluid portion 2₂ (FIG. 5a) mixes with the thickening agent, a speed of propagation of the second fluid portion 2₂ in the second permeable layer 13 may be reduced compared to a speed of propagation of a first fluid portion 2₁ in the first permeable layer 11. Put differently, this can allow a slower wicking rate in the second permeable layer 13 compared to a wicking rate in the first permeable layer 11.

Referring to FIG. 8, the first end region 4 of the test layer 3 may comprise a thickening agent 38 disposed between the first contact area 12 and the second contact area 14. This can help to decrease the flow rate of the second portion 2₂ in the first end region 4 relative to the flow rate of the first portion 2₁ in the first end region 4.

The thickening agent can also help to reduce back flow of the first fluid portion 2₁ in a direction away from the test region 3 towards the first end region 4. This can help to reduce mixing of the first fluid portion 2₁ and the second fluid portion 2₂ in a region outside the test region 6.

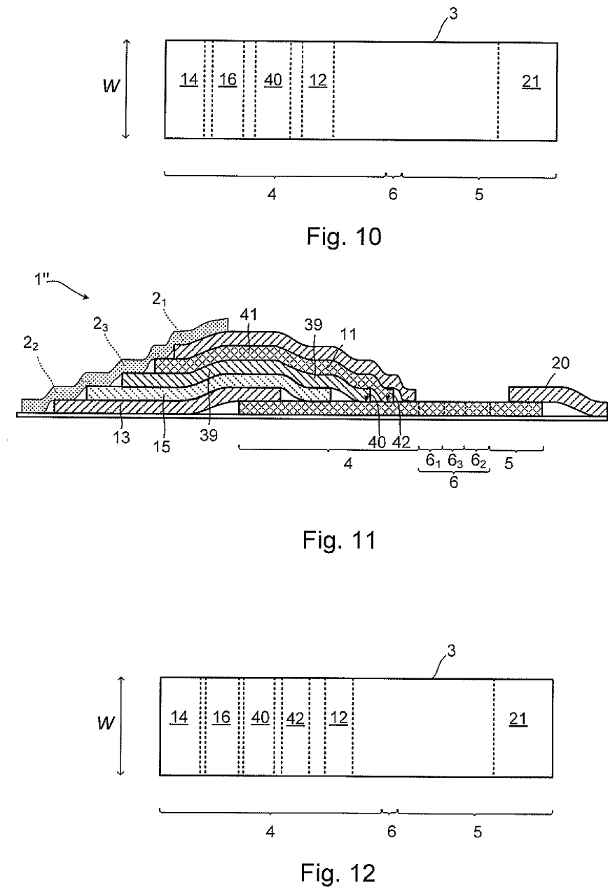

Referring to FIGS. 9 and 10, a first modified lateral flow device 1′ comprises the first, second, and third layers 11, 13, and 15 respectively, the test layer 3, the sample pad 17, and the absorbent pad 20. The modified lateral flow device 1′ further comprises a third permeable layer 39 between the first layer 11 and the third layer 15.

The third permeable layer 39 has a fourth contact area 40 with the test layer 6 on the first face 7. The fourth contact area 40 is between the third contact area 16 and the first contact area 12. The fourth contact area 4o is closer than the first contact area 12 to the second contact area 14.

The third permeable layer 39 is permeable to the liquid sample 2. The third permeable layer 39 comprises one or more permeable materials such as glass fibre, rayon, polyester.

The third permeable layer 39 may be thought of as a washing layer. In the lateral flow device 1, when a first portion 2₁ of fluid sample 2 propagates through the first permeable layer 11 and into the test layer 3, unbound analyte 22, enzyme-linked bound complexes 28, and enzyme-linked binding partners 23 may be deposited in the test layer 3 in regions outside the test region 6. When a second portion 2₂ of fluid sample 2 propagates through the second permeable layer 13 and into the test layer 3, deposited enzyme-linked binding partners 23 and enzyme-linked bound complexes 28 may react with substrate 26 carried by the second fluid portion 2₁. This can cause staining of the test layer 3 outside the test region 6.

In the first modified lateral flow device 1′, a third portion 2₃ of fluid sample 2 may propagate through the third permeable layer 39 and enter the test layer 3 at a point which is closer than the second contact area 14 to the absorbent pad 20. Thus the third fluid portion 2₃ may help to transport unbound analyte 22, enzyme-linked bound complexes 28, and enzyme-linked binding partners 23 deposited by the first fluid portion 2₁ in the first end region 4 further towards and through the test region 6. This can help to reduce background staining of the test layer 3 outside the test region 6.

Referring to FIGS. 11 and 12, a second modified lateral flow device 1″ comprises the first, second, and third permeable layers 11, 13, 39 respectively, the first non-permeable layer 15, the test layer 3, the sample pad 17, and the absorbent pad 20. The second modified lateral flow device 1″ further comprises a second non-permeable layer 41 between the third permeable layer 39 and the first permeable layer 11. The second non-permeable layer 41 is substantially non-permeable to the liquid sample 2.

The second non-permeable layer 41 has a fifth contact area 42 with the test layer 6 on the first face 7. The fifth contact area 42 is between the fourth contact area 36 and the first contact area 12.

This can help to prevent the first fluid portion 21 of fluid sample 2 from propagating into the third permeable layer 39 and contaminating the third permeable layer 39 with enzyme-linked bound complexes 28 and enzyme-linked binding partners 23.

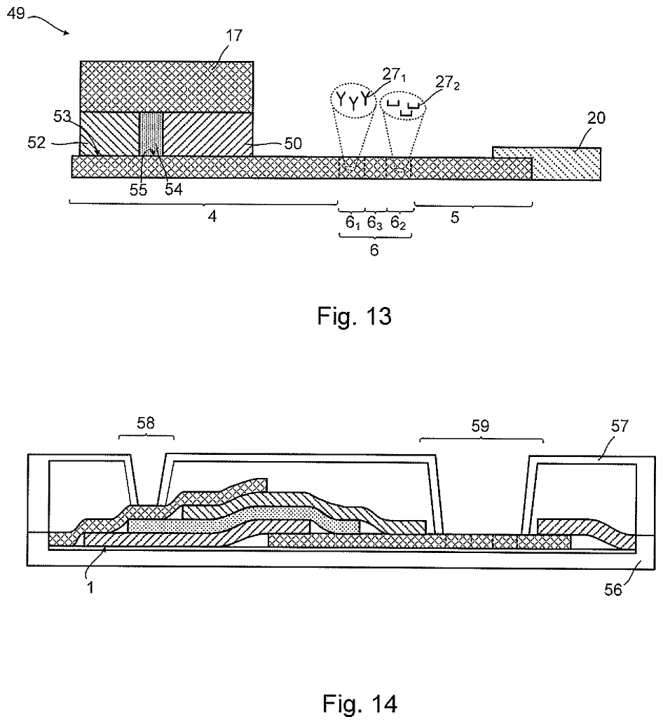

Referring to FIG. 13, a second lateral flow device 49 is shown. The device 49 comprises a test layer 3. The test layer 3 comprises first and second opposing end regions 4, 5 respectively, separated by a test region 6.

The test layer 3 has first and second opposite faces 7, 8 respectively and first and so second opposite ends 9, 10 respectively. The first and second opposite ends 9, 10 are in first and second opposite end regions 4, 5 respectively. The test layer 3 can hold and transport the liquid sample 2.

The device 49 comprises a first permeable layer 50 having a first contact area si in contact with the first face 7 in the first end region 4. The first contact area 51 is in contact with the first face 7 across the full width w of the test layer 3, that is, the width of the first contact area 51 is equal to w. The first contact area 51 is not in contact with the first end 9 of the test layer 3.

The first permeable layer 50 comprises one or more permeable materials such as glass fibre, rayon, polyester. The first permeable layer 50 contains an enzyme-linked binding agent 23 comprising a binding agent 24 which is linked, conjugated, or otherwise coupled to an enzyme 25. The binding agent 24 is capable of binding the analyte 22 specifically.

The first permeable layer 50 may include one or more materials which can perform one or more functions such as optimising a chemical reaction between the analyte 22 and the binding agent 24, stabilising the enzyme-linked binding agent 23, ensuring efficiency of release of the enzyme-linked binding agent 23 from the first layer so.

The device 49 comprises a second permeable layer 52 having a second contact area 53 in contact with the first end region 4. The second contact area 53 is in contact with the first face 7 across the full width w of the test layer 3, that is, the width of the first portion 14₁ is equal to w.

The second permeable layer 52 comprises one or more permeable materials such as glass fibre, rayon, polyester. The second permeable layer 52 contains an enzyme substrate 26. The enzyme substrate 26 is a substrate capable of reacting with the enzyme 25 linked to the binding agent 24 contained in the first layer 50. The second permeable layer 52 may include one or more materials which can perform one or more functions such as ensuring efficiency of release of the enzyme substrate 26 from the second layer 52, decreasing a propagation speed of a fluid through the second permeable layer 52 relative to a propagation speed of a fluid through the firs permeable t layer 50.

The device 49 comprises a first non-permeable layer 54 between the first layer so and the second layer 52. The first non-permeable layer 54 has a third contact area 55 with the first end region 4. The first contact area 51 and the second contact area 53 are separated by the third contact area 55. The first non-permeable layer 54 may comprise a plastic material such as HDPE, PTFE, PP, PVP, PVC, polyester, polystyrene.

The first contact area 51 is closer to the absorbent pad 20 than the second contact area 53 is to the absorbent pad 20.

The device 49 comprises a sample pad 17 in fluid contact with the first permeable layer 50 and the second permeable layer 52. The sample pad 17 comprises one or more absorbent materials such as cotton linter, high density cellulose, glass fibre, rayon, polyester. The sample pad 17 may include one or more of a pH buffer, a surfactant, a blocking reagent.

The device 1 comprises an absorbent pad 20 having a fourth contact area 21 in contact with the second end region 5. The device 1 comprises an absorbent pad 20 having a fourth contact area 21 in contact with the second end region 5. The absorbent pad 20 is in contact with the second end 10 of the test layer 3.

The test region 6 includes a first region 6₁ containing an immobilised specific binding agent 27₁. The immobilised binding agent 27₁ is capable of binding the analyte 22 specifically. For example, if the analyte 22 is an antigen, the immobilised binding agent 27₁ may be chosen to be an antibody which binds the antigen.

The test region 6 includes a second region 6₂, which may also be referred to as a control region, containing an immobilised common binding agent 27₂. The immobilised common binding agent 27₂ is capable of binding the enzyme-linked binding agent 23.

The first region 6₁ and the control region 6₂ are separated by a third region 6₃.

Referring to FIG. 14, a lateral flow device 1, 1′, 1″, 49 may be held in a housing. The housing comprises a base 56 and a lid 57. The lateral flow device 1 is received in the base 56 and the lid 57 is attached to the base 56 to secure the lateral flow device 1 and cover parts of the device 1 which do not require exposure. The lid 57 includes a sample receiving window 58 which exposes part of the sample pad 17. The lid 57 also includes a result viewing window 59 which exposes a part of the test region 6. The lid 57 and base 56 are made from a polymer such as, for example, polycarbonate, polystyrene, polypropylene or similar materials.

The sample receiving window 58 may expose a smaller or a larger part of the sample pad 17 than that shown in FIG. 14. This can allow control over the distribution of the fluid sample 2 (FIG. 5a) between first and second permeable layers 11, 13.

A lateral flow device 1, 1′, 1″, 49 may be fabricated by a roll-to-roll process or a sheet-by-sheet process. This can allow mass production of a lateral flow device.

It will be appreciated that various modifications may be made to the embodiments hereinbefore described. Such modifications may involve equivalent and other features which are already known in the design, manufacture and use of lateral flow devices and which may be used instead of or in addition to features already described herein. Features of one embodiment may be replaced or supplemented by features of another embodiment.

The first lateral flow device 1 or the second lateral flow device 49 may be configured to perform a ‘sandwich’ assay. For example, the first permeable layer 11 in the first lateral flow device 1 (or the first permeable layer 50 in the second lateral flow device 49) may comprise a primary binding agent capable of binding to the analyte 22. The second permeable layer 13 in the first lateral flow device 1 (or the second permeable layer 52 in the second lateral flow device 49) may comprise a secondary binding agent.

When the first fluid portion 2₁ propagates through the first permeable layer 11, the primary binding agent may bind to the analyte and the primary binding agent-analyte complex is transported to the test region 6 by the first fluid portion 2₁. The first fluid portion 2₁ may also transport unbound analyte to the test region 6. In the first region 6, of the test region 6 the unbound analyte may bind to the immobilised specific binding agent 27₁ and the bound analyte-primary antibody complex may bind to the immobilised specific binding agent 27₁.

The secondary binding agent is transported to the test region 6 by the second fluid io portion 2₂ which propagates through the second permeable layer 13. In the first test region 6₁ of the test region 6 the secondary binding agent may bind to a primary binding agent bound to an immobilised specific binding agent 27₁.

The first modified flow device 1′ or the second modified flow device 1″ may be configured to perform a sandwich assay. For example, the first layer 11 may comprise a primary binding agent capable of binding to the analyte 22. The fourth layer 39 may comprise a secondary binding agent linked to an enzyme. The second layer 13 may comprise a substrate.

When the first fluid portion 2₁ propagates through the first permeable layer 11, the primary binding agent may bind to the analyte to form a primary binding agent-analyte complex. The primary binding agent-analyte complex is transported to the test region 6 by the first fluid portion 2₁. The first fluid portion 2₁ may also transport unbound analyte to the test region 6. In the first region 6₁ of the test region 6 the unbound analyte may bind to the immobilised specific binding agent 27₁ and the bound analyte-primary antibody complex may bind to the immobilised specific binding agent 27₁.

The secondary binding agent linked to an enzyme is transported to the test region 6 by the third fluid portion 2₃ which propagates through the fourth layer 39. In the first test region 6₁ of the test region 6 the secondary binding agent may bind to a primary binding agent bound to an immobilised specific binding agent 27₁.

The substrate is transported to the test region 6 by the second fluid portion 2₂ which propagates through the second permeable layer 13. In the first region 6₁ of the test region 6, a reaction may occur between the substrate and an enzyme linked to a secondary binding agent, which may be bound to a primary binding agent bound to an immobilised specific binding agent 27₁.

The enzyme need not be linked to the binding agent directly.

For example, one or more enzymes may be linked to a carrier particle and the binding agent may be linked to the carrier particle.

The carrier particle may be a metal or carbon-based nanoparticle such as a gold or silver nanoparticle or a carbon nanotube. The carrier particle may be a polymer such as as Poly(N-isopropylacrylamide) or an avidin or streptavidin crosslinked polymer. The carrier particle may be a peptide such as polylysine, elastin like-polypeptide, polyaniline.

The binding agent may be a DNA, RNA, or PNA probe molecule. The binding agent may be a peptide or nucleic acid aptamer.

The immobilised specific binding agent need not be an antibody. The immobilised specific binding agent may be a protein, for example, protein A or protein G or a biotin binding protein such as streptavidin or avidin. The first permeable layer may then comprise a first binding agent, such as an antibody, capable of binding to the analyte and a second binding agent capable of binding to the analyte or to the first binding agent. The second binding agent is capable of binding to the protein immobilised at the test region. The second binding agent may be a capture antibody or biotin-modified capture antibody or other biotin-modified binding agent. The first binding agent may be linked to an enzyme or other reporter, such as a gold nanoparticle, as described herein.

The device may be used to deliver one or more substances dried on the permeable layers to the test region at different times. The substances need not include an enzyme or a substrate.

For example, the first permeable layer may comprise a binding partner linked to a gold or a silver nanoparticle and the second permeable layer may comprise a gold- or silver-enhancing solution for increasing the size of the nanoparticles and thus the optical absorption coefficient or the molar extinction coefficient of the modified nanoparticles. The enhancing solution may comprise gold or silver ions, Danscher solution (gum arabic-silver lactate-hydroquinone enhancement solution), light insensitive (LI) Silver, high quality (HQ) Silver. The enhancing solution may comprise an initiator solution and a gold or silver salt solution.

The lateral flow devices herein described allow the enhancing solution to be delivered to the test region after the nanoparticle has been delivered to the test region.

One or more of the non-permeable layers may be coated with adhesive in order to facilitate assembly. The adhesive may be adhesive tape.

The device 1, 1′, 1″ may be provided without the housing. The device 1, 1′, 1″ may be held together using adhesive tape. The device 1, 1′, 1″ may include an external protecting layer of adhesive tape.

The device 1, 1′, 1″ may be fabricated by a lamination process. The first, second, and third permeable layers 11, 13, 39 and the first and second non-permeable layers 15, 41, the sample pad 17, and the absorbent pad 20 may be flexible.

1. A lateral flow device comprising:
a test layer comprising a test region between opposite first and second end regions of the test layer;
a first permeable layer having a first contact area in contact with the first end region;
a second permeable layer having a second contact area in contact with the first end region;
a first non-permeable layer between the first and second permeable layers and having a third contact area with the first end region, wherein the first contact area and the second contact area are separated by the third contact area; and
a sample pad in fluid contact with the first and second permeable layers for delivery of a sample fluid to the first and second contact regions;
wherein the second contact area is further from the second end region than the first contact area is from the second end region.

2. A device according to claim 1, wherein at least a portion of the first non-permeable layer is interposed between the first permeable layer and the second permeable layer.

3. A device according to claim 1, wherein at least a portion of the second permeable layer is disposed on the non-permeable layer.

4. A device according to claim 1, further comprising an absorption region in fluid contact with the second end region of the test layer.

5. A device according to claim 1, wherein the test layer has first and second opposing ends disposed in first and second end regions respectively.

6. A device according to claim 5, wherein the first permeable layer is in direct contact with the first end.

7. A device according to claim 1, wherein the first permeable layer comprises a binding agent linked to a carrier particle.

8. A device according to claim 7, wherein the carrier particle is a gold nanoparticle.

9. A device according to claim 7, wherein the second permeable layer comprises a gold-enhancing solution.

10. A device according to claim 7, wherein an enzyme is linked to the binding agent.

11. A device according to claim 7, wherein an enzyme is linked to the carrier particle.

12. A device according to claim 11, wherein at least two enzymes are linked to the carrier particle.

13. A device according to claim 12, wherein the second permeable layer comprises an enzyme substrate.

14. A device according to claim 9, wherein the second permeable layer comprises a first region proximal to the second contact area and a second region distal to the second contact area and wherein a relatively high concentration of gold-enhancing solution or enzyme substrate is disposed in the second region and a relatively low concentration of gold-enhancing solution or enzyme substrate is disposed in the first region.

15. A device according to claim 1, wherein the test region comprises an immobilised specific binding agent.

16. A device according claim 15, wherein the test region comprises first and second regions and the immobilised specific binding agent is disposed in the first region and an immobilised common binding agent is disposed in the second region.

17. A device according to claim 1, wherein the test layer comprises a thickening agent for slowing fluid flow disposed between the first contact area and the second contact area.

18. A device according to claim 1, wherein a path length of the sample fluid within the first permeable layer is not equal to a path length of the sample fluid within the second permeable layer.

19. A device according to claim 1, wherein a path length of the sample fluid within the first permeable layer is equal to a path length of the sample fluid within the second permeable layer.

20. A device according to claim 1, further comprising:
a third permeable layer disposed between the first permeable layer and the first non-permeable layer, the third permeable layer having a fourth contact area in contact with the first end region, wherein the third contact area is between the first contact area and the second contact area.

21-32. (canceled)
